(19) United States
(12) Patent Application Publication
(10) Pub. No.: US 2007/0174084 A1
(43) Pub. Date: Jul. 26, 2007

(54) System and method for connecting individuals with remote volunteer medical personnel

(76) Inventors: Stuart O. Goldman, Scottsdale, AZ (US); Richard E. Krock, Naperville, IL (US); Bernard L. Malone III, Little Rock, AR (US); Karl F. Rauscher, Emmaus, PA (US); James P. Runyon, Wheaton, IL (US)

Correspondence Address: Richard J. Minnich, Esq.;Fay, Sharpe, Fagan, Minnich & McKee, LLP, Seventh Floor, 1100 Superior Avenue, Cleveland, OH, 44114-2579, US

(21) Appl. No.: 11/337,891

(22) Filed: Jan. 23, 2006

(51) Int. Cl. G06Q 99/00; G06Q 10/00; A61B 5/00

(52) U.S. Cl. 705/2; 600/300; 705/1

(57) ABSTRACT

A method of providing an individual seeking medical attention (20) access to a remotely located volunteer medical professional (40) includes: screening the individual (20) to determine a type of medical attention that is called for; searching through a plurality of volunteer medical professionals having diverse medical qualifications; selecting a volunteer from the search that is suited to provide the type of medical attention that is called for as determined from the screening, the volunteer being selected based upon their medical qualifications; and, establishing a connection over a telecommunications network (50) between the individual (20) and the selected volunteer (40) such that they are able to interactively communicate with one another over the established connection.

### FIELD

The present inventive subject matter relates to the telecommunication arts. One particular application is found in conjunction with a remote volunteer telecommunication (RVT) service and/or system for use by individuals in a catastrophe stricken or other like region, and the specification makes particular reference thereto. However, it is to be appreciated that aspects of the present inventive subject matter are also amenable to other like applications.

### BACKGROUND

In times of natural disaster or other like catastrophes, people in the effected area may be unable to use normal or otherwise conventional channels to obtain medial attention, be it emergency medical attention or relatively routine medical attention. For example, insomuch as there is generally a limited number of medical. professionals or personnel (i.e., doctors, dentists, nurses, paramedics, pharmacists, veterinarians, etc.) normally present in an area when a catastrophe strikes, these medical professionals may be over extended and hence unavailable to provide medial attention to all the individuals seeking it at that time, particularly in light of the fact that during a relatively widespread catastrophe (e.g., city wide) the number of people seeking medical attention is often significantly elevated. Likewise, ambulance services, paramedic services, medical facilities (e.g., hospitals, emergency rooms, urgent care centers, medial offices, pharmacies, etc.) and/or other similar medical resources in the effected region may also be overcrowded or overloaded or otherwise unavailable due to the catastrophe. Additionally, in some cases large numbers of the population may be physical displaced, thus further severing their ties with their historic medical providers.

Traditionally, to address the shortage of medial professionals commonly experienced as the result of a catastrophic event, volunteers from outside the effected area are recruited, trained in the specifics of the situation and transported to the effected region. This approach, however, has some limitations and/or drawbacks. First, it takes time to recruit willing volunteers, even more time to train them and even more time to transport them to the effected region. To the extent that a given volunteer mobilization effort is merely reactive to a particular catastrophe as opposed to a proactive solution, the aforementioned time that it takes to recruit, train and/or transport volunteers represents a delay in the response. In a catastrophe situation, significant time delays for making volunteers available to the effected region are generally undesirable. Second, to justify the amount of training and transportation costs that are typically associated with such a mobilization, volunteers are generally obligated to make a significantly long term time commitment to the volunteer effort, e.g., the commitment may be a continuous block of time on the order of a couple of weeks or more. As many medical professionals have other jobs and/or obligations in their respective home regions, they may not be willing to or may not be able to provide such a commitment. Accordingly, those individuals that may otherwise be available for some shorter time periods or intermittently available are potentially excluded or dissuaded from volunteering.

Accordingly, a new and improved telecommunications service and/or system is disclosed that overcomes the above-referenced problems and others.

### SUMMARY

In accordance with one embodiment, a method of providing an individual seeking medical attention access to a remotely located volunteer medical professional includes: screening the individual to determine a type of medical attention that is called for; searching through a plurality of volunteer medical professionals having diverse medical qualifications; selecting a volunteer from the search that is suited to provide the type of medical attention that is called for as determined from the screening, the volunteer being selected based upon their medical qualifications; and, establishing a connection over a telecommunications network between the individual and the selected volunteer such that they are able to interactively communicate with one another over the established connection.

In accordance with another embodiment, a system is provided for connecting an individual seeking medical attention to a remotely located volunteer medical professional. The system includes: access means by which an individual seeking medical attention selectively accesses the system; screening means which screen the individual accessing the system to determine a type of medical attention that is called for; data storage means containing identities of a plurality of volunteer medical professional along with each volunteer's medical qualifications; searching means for searching the data storage means to select a volunteer from the search that is suited to provide the type of medical attention that is called for by the screening means, the volunteer being selected based upon their medical qualifications; and, connecting means for establishing a connection over a telecommunications network between the individual and the selected volunteer such that they are able to interactively communicate with one another over the established connection.

Numerous advantages and benefits of the inventive subject matter disclosed herein will become apparent to those of ordinary skill in the art upon reading and understanding the present specification.

### BRIEF DESCRIPTION OF THE DRAWINGS

The inventive subject matter may take form in various components and arrangements of components, and in various steps and arrangements of steps. The drawings are only for purposes of illustrating preferred embodiments and are not to be construed as limiting. Further, it is to be appreciated that the drawings are not to scale.

FIG. 1 is a block diagram illustrating an exemplary RVT system that embodies aspects of the present inventive subject matter.

FIG. 2 is a flow chart illustrating an exemplary process for registering an individual as a volunteer in accordance with aspects of the present inventive subject matter.

FIG. 3 is a block diagram illustrating an exemplary configuration of an RVT system suitable for practicing aspects of the present inventive subject matter.

FIG. 4 is a flow chart illustrating an exemplary operation of an RVT system in accordance with aspects of the present inventive subject matter.

### DETAILED DESCRIPTION

For clarity and simplicity, the present specification shall refer to structural and/or functional elements, entities and/or facilities, relevant communication standards, protocols and/or services, and other components and features that are commonly known in the telecommunications art without further detailed explanation as to their configuration or operation except to the extent they have been modified or altered in accordance with and/or to accommodate the embodiment(s) presented herein.

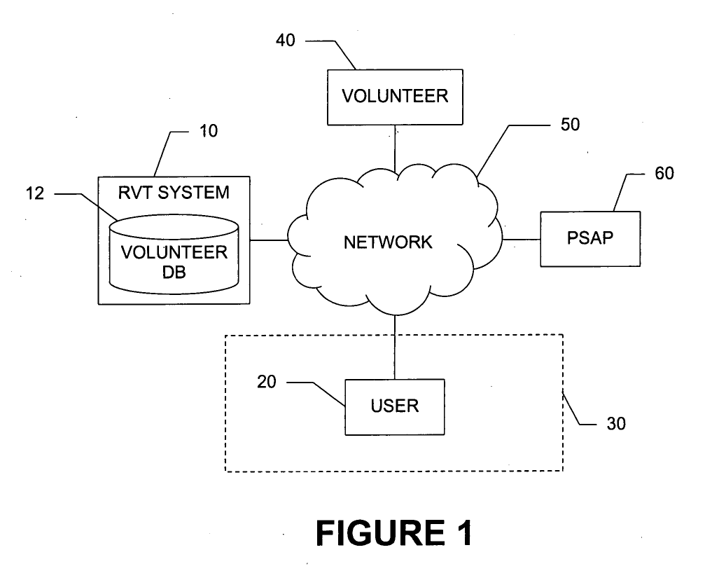

With reference to FIG. 1, a RVT system 10 provides an individual or user 20 (e.g., in a catastrophe stricken area or other geographic region 30) access to a volunteer 40 (e.g., a medical professional such as a doctor, a dentist, a nurse, a paramedic, a pharmacist, a chiropractor, an ophthalmologist, an optometrist, a psychologist, a psychiatrist, a veterinarian, etc.) that is remotely located (e.g., outside the region 30). Optionally, the user 20 is an individual that has had ties with their customary medical provides severed for one reason or another, e.g., an individual that is displaced from their normal geographic region due to a catastrophe. In the illustrated example, only one user 20 and one volunteer 40 are shown for simplicity and clarity. However, it is to be appreciated that in practice generally a plurality of users and/or volunteers are similarly situated and/or served by the system 10. Additionally, while only one localized system 10 is shown in the illustrated example, optionally, the system 10 is distributed and/or mirrored in a plurality of different sites, different locations and/or different network nodes to promote the reliability and/or availability of the system 10. Moreover, it is to be appreciated that the user 20 may in fact be the patient or the actual individual seeking medical attention for himself, or alternately, the user 20 may be someone (e.g., a first responder) that is attending to the actual patient, i.e., someone who is seeking medical attention for another individual.

Suitably, the RVT system 10 provides a RVT service or feature to one or more users and is implemented as one or more applications or software programs or other appropriate collection of instructions running on a platform and/or being executed by a server, computer or other suitable hardware to perform and/or administer the tasks, processes and/or functions associated with the RVT service or feature described herein. While only a single server optionally implements the RVT system 10, it is to be appreciated that alternately one or more servers or other hardware or components or subsystems may act separately or in combination to function as web servers, application servers, database servers, and/or other components and/or subsystems, such that collectively they operate in conjunction with one another to implement, administer and/or support the RVT system 10.

As shown, the RVT system 10, the user 20 and the volunteer 40 are operatively connected to a telecommunications network 50 in the usual manner so as to enable communication between the aforementioned entities. Suitably, the network 50 takes the form of a public switched telephone network (PSTN), the Internet, or some other like network or some combination thereof, depending on the types of end user terminals (EUTs) being employed by the respective parties 20 and 40 (e.g., telephones, general purpose computers, personal digital assistants (PDAs), etc.) and/or the communication modes being employed by the respective parties 20 and 40 (e.g., voice, data, image, video, short message service (SMS), instant messaging, test messaging, e-mail, etc.).

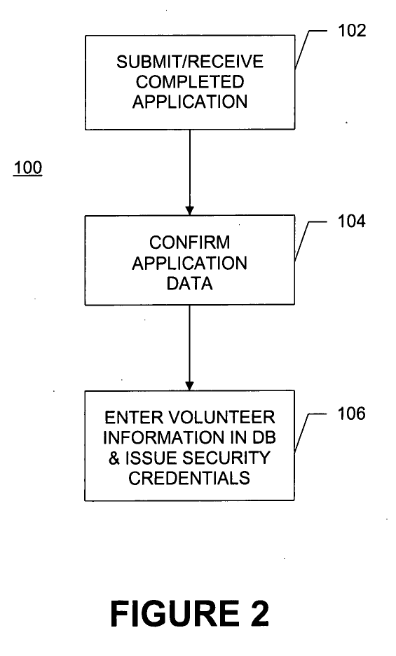

With reference to FIG. 2, a flow chart illustrates an exemplary process 100 by which a medical professional or other individual registers with the RVT system 10 to be a volunteer 40. Suitably, the process begins at step 102 with the registering individual submitting and/or the RVT system 10 receiving an appropriate completed application. For example, the completed application includes: the identity of the individual registering, their contact information (i.e., home and/or office address, e-mail address, telephone number, etc.), their qualifications (i.e., type of profession or license, kind of education, training or degree, area of practice or specialty, etc.), any restrictions relating to their qualifications and/or capabilities, etc. Suitably, the application is submitted to and/or received by the RVT system 10 over the network 50. For example, using a general purpose computer or other Internet enabled EUT, the individual registering selectively accesses the RVT system 10 over the Internet and enters the requested information to complete the application provided by the system 10.

At step 104, the application information is confirmed to ensure that an applicant is legitimate and qualified. For example, the identity and/or contact information is verified. Optionally, government and/or other regulatory agencies or bodies for the various professions are checked to see that any indicated licenses are in order and/or that the individual is otherwise in good standing. At step 106, assuming the individual is legitimate and suitably qualified, the application information is entered, stored and/or otherwise maintained in a volunteer database (DB) 12 (see FIG. 1) or some other suitable file or other appropriate data storage location incorporated in and/or otherwise accessible by the RVT system 10, and a user ID and password or other like security credentials are established and/or issued to the individual. Once received, the security credentials are optionally used by registered volunteers (like the volunteer 40) to selectively login or sign-on to the RVT system 10.

Notably, the RVT system 10 provides a proactive solution to medical personnel shortages, e.g., resulting from catastrophic events. That is to say, medial personnel and/or other individuals may selectively register at any time, e.g., even well before a specific catastrophic event occurs that might otherwise prompt their particular participation in a volunteer effort. Accordingly, any appropriate training and/or instructions for using the RVT system 10 or in general for providing volunteer medical services can be conducted and/or provided beforehand. In this manner, the pre-trained volunteers are prepared and/or ready for service in advance of a given catastrophe and can take action as soon as is practical after the catastrophe hits, i.e., without training delays.

Suitably, when the volunteer 40 desires to begin volunteering, they simply login or sign-on to the RVT system 10 from a convenient location of their choosing, e.g., from their home location which may be remote from the catastrophe or otherwise outside the region to which they are providing volunteer services. Conversely, when the volunteer 40 is done or wishes to quit volunteering, they simply logout or sign-off of the RVT system 10. In this way, the volunteer 40 may selectively choose and/or regulate their own schedule while still providing valuable volunteer services from a remote location. Suitably, the system 10 recognizes which volunteers are logged-in and which are not at any given time. Optionally, the current status of each volunteer in the DB 12 is noted or recorded in the DB 12 along with that volunteer's other information. For example, this status is update each time the volunteer 40 logs-in and/or logs-out.

Suitably, the volunteer 40 employs a general purpose computer or other Internet enabled EUT to connect to and/or communicate with the RVT system 10 over the Internet or another like network, e.g., using a high-speed or broadband connection. The high-speed or broadband connection facilitates a richer communication and/or exchange of information, e.g., simultaneously supporting a combination of one or more communication modes, such as voice, image, video, text and/or data. Alternately, however, it is contemplated that a lower bandwidth connection may be used. Moreover, if the volunteer 40 does not have access to a computer or Internet connection they may choose to be “on call” so that the RVT system 10 may call or contact the volunteer 40 on their telephone or other similar EUT when they are deemed a suitable match for a user seeking medical attention, e.g., such as the user 20.

Generally, when the user 20 desires to seek medial attention, they employ an EUT to call or access the RVT system 10 over the network 50. In response, the system 10 optionally performs an automated triage and/or initial screening process through which the user 20 is passed, e.g., to initially determine the nature of the medical attention that is appropriate, the symptoms, the degree of severity of the condition and/or a potential diagnosis. Accordingly, based upon the outcome of the triage and/or initial screening, the RVT system 10 identifies a currently logged-in volunteer (e.g., from the DB 12) that is best suited to provide the appropriate medical attention to the particular user 20. The user 20 is then connected to the identified volunteer, e.g., the volunteer 40. In this manner, the user is matched with the most suitable volunteer available to assist with and/or address the user's particular problem. That is to say, e.g., if the user is seeking medical attention for a dental problem, then the triage or screening process identifies this problem and the system 10 scans the DB 12 to find an available dentist that is currently logged-in; or if the user is seeking medial attention for a vision or eye related problem, then the triage or screening process identifies this problem and the system 10 scans the DB 12 to find an available ophthalmologist or optometrist that is currently logged-in; or if the user is seeking medial attention for a heart related problem, then the triage or screening process identifies this problem and the system 10 scans the DB 12 to find an available cardiologist that is currently logged-in; or if the user is seeking medial attention for an emergency or trauma related injury, then the triage or screening process identifies this problem and the system 10 scans the DB 12 to find an available emergency or trauma specialist that is currently logged-in; or if the user is seeking medial attention for a non-specific and/or non-emergency related medical problem, then the triage or screening process identifies this problem and the system 10 scans the DB 12 to find an available general practitioner that is currently logged-in; and so on. Optionally, if an ideal or exact match for a user cannot be found among the volunteers currently logged-in, then the system 10 progresses through a succession of next most desirable or suitable options to find as good a match of volunteer as possible, i.e., operating on the principal that some match is at least better than none at all.

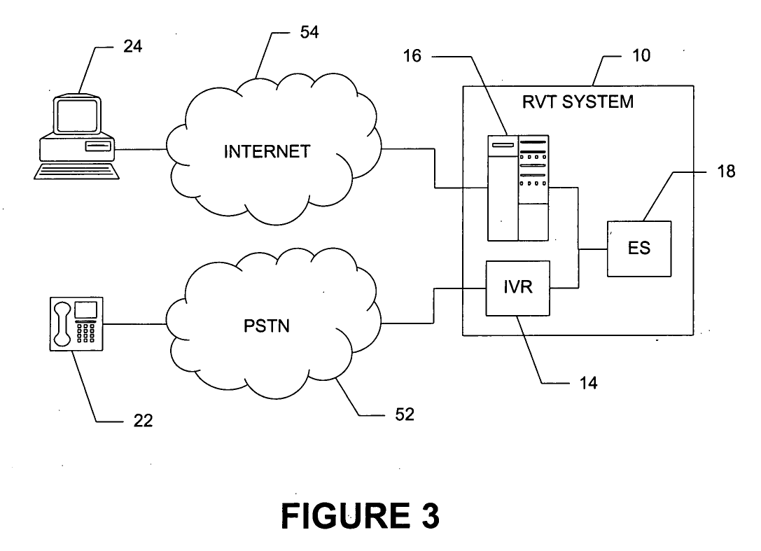

With reference to FIG. 3, suitably the RVT system 10 supports and/or is accessible by different users via a variety of communication modes and/or EUTs. For example, one user may employ a telephone 22 to place a voice call to the RVT system 10 over the PSTN 52. Accordingly, the system 10 is optionally equipped or otherwise provisioned with an interactive voice response (IVR) system 14 that is used to collect input from and/or provide output to the user. Alternately, another user may employ a general purpose computer or other Internet enabled EUT 24 to access the RVT system 10 over the Internet 54. Optionally, the user employs a high-speed or broadband connection to facilitate a richer communication and/or exchange of information, e.g., simultaneously supporting a combination of one or more communication modes, such as voice, image, video, text and/or data. Alternately, however, it is contemplated that a lower bandwidth connection may be used. In either case, the system 10 is optionally equipped or otherwise provisioned with a web, application or other server 16 that is used to collect input from and/or provide output to the user. Suitably, in the case of a lower bandwidth Internet connection, the user may opt to also place a voice call to the system 10. The system 10 would then associate the two feeds (i.e., one from the Internet connection and one from the voice call) with one another, e.g., receiving data and the like via the Internet connection and audio via the voice call.

The RVT system 10 is also optionally equipped or otherwise provisioned with what is known as an expert system (ES) 18 or other like artificial intelligence or similar application, function or service that conducts and/or regulates the triage and/or initial screening process. Suitably, the ES 18 conducts the triage and/or initial screening process by collecting or otherwise obtaining pertinent and/or relevant information or data from the user 20 about their condition or the reason they called or accessed the system 10. For example, a standard medical screening tree is followed. Optionally, the information is collected via a series of questions posed to and answered by the user 20 which are intended and/or designed to elicit the desired information, such as, e.g., personal information about the user or patient (i.e., age, weight, height, sex, etc.), their medical background (i.e., known medical conditions, allergies, history of medical problems, etc.), the current symptoms, any known vital statistics (i.e., pulse rate, blood pressure, temperature, etc.), the type and/or location of any injury, the amount or degree of pain if any, the cause and/or circumstances surrounding any accident or trauma involved, medications currently being taken or other treatment already received, etc.

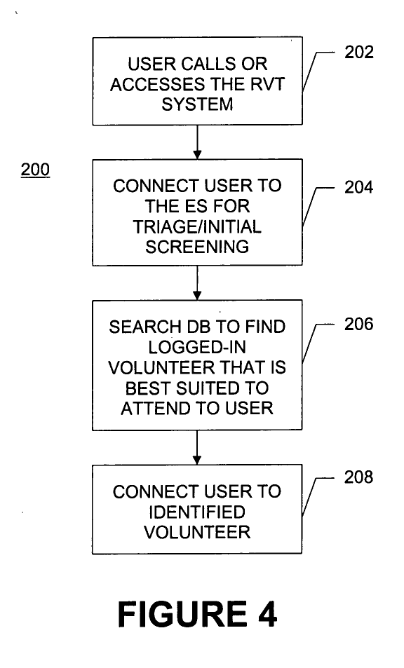

Turning attention to FIG. 4, the operation of the RVT system 10 is now described with reference to an exemplary process 200. As shown the process 200 begins at step 202 with the user 20 calling or otherwise accessing the RVT system 10, e.g., over the network 50. In response, at step 204, the RVT system 10 connects the user 20 to the ES 18 or other like application, function or service that conducts the triage and/or initial screening process. Suitably, based upon the user's input or responses to questions posed during the triage and/or initial screening process, the ES 18 develops or generates a medical evaluation, potential diagnosis, suggested treatment, recommended course of action and/or other like medical conclusions, which are optionally provided or output to the user 20 by the system 10. For example, the system 10 and/or the ES 18 optionally calculates or otherwise determines the probable accuracy of the conclusions reached by the ES 18, and if the probability of accuracy exceeds a sufficient threshold, then the conclusions are provided to the user 20, otherwise they are not. Alternately, the user 20 is given the option to receive or not receive the conclusions generated by the ES 18. Even if the conclusions generated by the ES 18 are output to the user 20, suitably, the system 10 still provides the user 20 with the option of being connected to a volunteer if they so desire.

Assuming the user 20 still desires to be connected with a volunteer, at step 206, the RVT system 10 searches the DB 12 to find an available logged-in volunteer that is best suited. to attend to the user 20. That is to say, based upon the results of the triage or initial screening process and/or the information collected thereby, the system 10 searches the DB 12 for an available logged-in volunteer that is the type of professional and/or otherwise has qualifications that are most compatible with the type of medical attention deemed appropriate for the user 20. Optionally, if the ideal volunteer or an exact fit cannot be found among the volunteers currently logged-in and available (i.e., not already attending to another user), then the system 10 progresses through a succession of next most desirable or suitable options to find as good a match of volunteer as possible. For example, if the user 20 had a vision or eye problem, then the best fit or qualified volunteer may be an ophthalmologist or optometrist; however, if no such volunteer where logged-in or available at the time, then a second best choice may be a general practitioner; and if no general practitioner is logged-in or available at the time, then the third best choice may be a nurse; and so on.

In the case that no suitable volunteer is logged-in and/or available, optionally, the system 10 reports this fact to the user 20 and advises them to try again later. Of course, the system 10 optionally still provides them automated conclusions, e.g., generated by the ES 18. Alternately, if otherwise suitable volunteers are in fact logged-in but merely busy with prior connected users, the system 10 may provide the current user with an option to wait or hold for a volunteer. If the user chooses to exercise this option, they are placed in a queue so as to be connected with the next suitable volunteer in turn when they become available. Likewise, the system 10 optionally provides users the ability to hold or wait for a suitable volunteer to login. Suitably, the system 10 is also provisioned to take corrective steps or measures if a shortage of volunteers is detected at any given time. For example, if a shortage is detected, the system 10 is optionally programmed to automatically contact volunteers (e.g., via e-mail, automated telephone calls, etc.) which are not logged-in to the system 10 and request that they do login and/or inform them of the shortage. Suitably, the volunteers contacted in this manner are selected from the DB 12 in response to the particular types of profession or kinds of qualifications where the shortage is being experienced.

Assuming the volunteer 40 is logged-in, available (i.e., not already connected to another user), and has been identified in step 206 as a suitable match or fit for the user 20, then the user 20 is connected to the volunteer 40 at step 208. Accordingly, the user 20 and volunteer 40 are free to actively communicate with one another. For example, the volunteer 40 is able to communicate with the user 20 to facilitate the medical evaluation of the user or patient, to make a diagnosis or confirm or replace a diagnosis made by the ES 18, to reassure the user 20, to prescribe treatment or suggest a recommended course of action, etc. Suitably, in conjunction with connecting the user 20 to the volunteer 40, the RVT system 10 optionally provides the volunteer 40 with selected information and/or data collected during the triage and/or initial screening process, and optionally also provides the volunteer 40 with one or more of the conclusions reached by the ES 18 (if any).

Suitably, the type of connection and/or the capabilities of the EUTs being employed by the user 20 and/or the volunteer 40 dictate or influence the communication modes used between the RVT system 10 and the respective parties. Optionally, each party's connection type, the communication modes and/or the capabilities of their EUTs are factors in determining which volunteer is the best match or fit for a given user, i.e., the better the compatibility between the foregoing then the better the match. For example, to maximize the rich communication potential of high-speed or broadband Internet connections, there is suitably a preference for connecting a user accessing the system 10 via a high-speed or broadband connection with a volunteer that is logged-in to the system 10 via a high-speed or broadband connection. Optionally, to achieve a desired balance, the system 10 applies selected weighting to the factors used in the matching making decision, e.g., the two factors that are weighted accordingly are (i) the compatibility between the volunteer's qualifications and the type of medical attention called for by the user, and (ii) the commonality of communication modes or capabilities. Suitably, a more complex equation or calculation is optionally used to balance the factors so as to achieve the desired performance.

As can be appreciated, when the user 20 and the volunteer 40 are both connected to the RVT system 10 in a similar manner and/or using similar communication modes, then establishing the connection between the parties is relatively simple. For example, if the user 20 employs a computer or other Internet enabled EUT to access the RVT system 10 over the Internet, then the RVT system 10 optionally provides the user 20 a webpage, window or other screen in which to enter the data or information collected during the triage or initial screening process. Similarly, if the volunteer 40 also employs a computer or other Internet enabled EUT to login to the RVT system 10 over the Internet, then the RVT system 10 optionally provides the volunteer 40 a similar webpage, window or other screen populated with the collected data or information entered by the user 20 and optionally including any conclusions reached by the ES 18. Depending on the bandwidth of the connections and/or the communication modes supported by the respective EUTs being used, the parties 20 and 40 are given the option to select one or more interactive communication modes. For example, in a low bandwidth situation, a dialog box or the like is optionally provided to both the parties 20 and 40 so that they can exchange text messages therein, e.g., via instant messaging or an Internet chat session conducted between the parties. In a higher bandwidth situation, the user 20 and/or the volunteer 40 are optionally provided a “click-to-talk” button or other similar link or object. When this option is selected, the RVT system 10 establishes, e.g., a VOIP (Voice over Internet Protocol) session or other like connection between the parties 20 and 40 so that they can speak to one another. Similarly, the RVT system 10 optionally supports the transmission or exchange of video and/or images between the parties.

Suitably, for other instances or occasions where the communication modes used by the parties 20 and 40 are not the same, the RVT system 10 is also equipped or otherwise provisioned to translate between different yet compatible communication modes. For example, the user 20 may optionally have placed a voice call to the RVT system 10 (i.e., as shown in FIG. 3, the user employed the EUT or telephone 22 to call the RVT system 10 over the PSTN 52). Accordingly, the IVR 14 is employed by the system 10 to collect the user's input and/or responses to questions posed during the triage and/or initial screening process conducted by the ES 18. Assuming the volunteer 40 in this example logs-in the same way as in the prior example (i.e., employing a computer or other Internet enabled EUT to login to the RVT system 10 over the Internet), then the RVT system 10 optionally provides the volunteer 40 the same or a similar webpage, window or other screen as in the prior example. However, before the system 10 populates the volunteer's screen with the collected data or information entered by the user 20, the system 10 first converts the user's vocal or DTMF (Dual Tone Multi-Frequency) inputs into corresponding text and/or data. The interactive communication between the parties in this case is optionally established on one end via the otherwise conventional voice call between the user 20 and the RVT system 10, and on the other end via a VolP or other like session between the volunteer 40 and the system 10. Suitably, the system 10 acts as a gateway between the Internet or IP portion of the connection and PSTN portion of the connection. Of course, it is to be appreciated that the foregoing is just one particular example, and that in. practice the system 10 is suitably provisioned to perform other similar translations or conversions between other different communication modes, different protocols, and the like in one or both traffic directions (i.e., from user to volunteer and vice versa from volunteer to use).

Since the system 10 is likely to be accessed by user in catastrophe stricken areas, there is the possibility that normal communication infrastructure (e.g., telephone lines, cellular towers or base stations, telecommunication switches, etc.) may be damaged or out-of-order. In such instances, it may be beneficial to employ telecommunication channels that are optionally routed and/or augmented by satellite feeds or transmissions, e.g., using one or more mobile satellite truck or -vans that brought into the effected area after the catastrophe. Additionally, since people traditionally call 9-1-1 in emergency situation commonly associated with catastrophes, optionally the pubic safety answering point (PSAP) 60 serving the effected area is interfaced with the RVT system 10 (see FIG. 1). Suitably, the interface is the same or similar to that conventionally used between the PSAP and traditional first responders (i.e., police, fire, ambulance, etc.). This feature is particularly valuable when the 9-1-1 system remains intact and/or functional, but ambulance and/or other like medical services have been compromised.

It is to be appreciated that in connection with the particular exemplary embodiments presented herein certain structural and/or function features are described as being incorporated in defined elements and/or components. However, it is contemplated that these features may, to the same or similar benefit, also likewise be incorporated in other elements and/or components where appropriate. It is also to be appreciated that different aspects of the exemplary embodiments may be selectively employed as appropriate to achieve other alternate embodiments suited for desired applications, the other alternate embodiments thereby realizing the respective advantages of the aspects incorporated therein.

It is also to be appreciated that particular elements or components described herein may have their functionality suitably implemented via hardware, software, firmware or a combination thereof. Additionally, it is to be appreciated that certain elements described herein as incorporated together may under suitable circumstances be stand-alone elements or otherwise divided. Similarly, a plurality of particular functions described as being carried out by one particular element may be carried out by a plurality of distinct elements acting independently to carry out individual functions, or certain individual functions may be split-up and carried out by a plurality of distinct elements acting in concert. Alternately, some elements or components otherwise described and/or shown herein as distinct from one another may be physically or functionally combined where appropriate.

In short, the present specification has been set forth with reference to preferred embodiments. Obviously, modifications and alterations will occur to others upon reading and understanding the present specification. It is intended that the invention be construed as including all such modifications and alterations insofar as they come within the scope of the appended claims or the equivalents thereof.

What is claimed is:

1. A method of providing an individual seeking medical attention access to a remotely located volunteer medical professional, said method comprising:
(a) screening the individual to determine a type of medical attention that is called for;
(b) searching through a plurality of volunteer medical professionals having diverse medical qualifications;
(c) selecting a volunteer from the search that is suited to provide the type of medical attention that is called for as determined from the screening, said volunteer being selected based upon their medical qualifications; and,
(d) establishing a connection over a telecommunications network between the individual and the selected volunteer such that they are able to interactively communicate with one another over the established connection.

2. The method of claim 1, wherein the screening of step (a) comprises:
posing a series of medical questions to the individual; and,
collecting responses to the questions from the individual, wherein the type of medical attention that is called for is determined based upon the collected responses.

3. The method of claim 2, further comprising:
communicating the collected responses to the selected volunteer to which the individual is connected.

4. The method of claim 2, further comprising:
automatically developing a diagnosis based upon the collected responses; and,
communicating the diagnosis to at least one of the individual and the selected volunteer to which the individual is connected.

5. The method of claim 1, further comprising:
maintaining a database identifying the plurality of volunteers along with their medical qualification, wherein the searching of step (b) is conducted in said database.

6. The method of claim 5, wherein the database further includes contact information for each volunteer.

7. The method of claim 1, wherein each of the plurality of volunteers selectively enables and disables their own eligibility to be selected in step (c).

8. The method of claim 1, further comprising:
providing a site accessible to the plurality of volunteers via the telecommunications network, wherein each of the plurality of volunteers selectively logs-in and logs-out of the site such that when step (c) is execute volunteers that are currently logged-in are eligible for selection and volunteers that are currently logged-out are not eligible for selection.

9. The method of claim 1, wherein the telecommunications network is an Internet Protocol (IP) network.

10. A system for connecting an individual seeking medical attention to a remotely located volunteer medical professional, said system comprising:
access means by which an individual seeking medical attention selectively accesses the system;
screening means which screen the individual accessing the system to determine a type of medical attention that is called for;
data storage means containing identities of a plurality of volunteer medical professional along with a record of each volunteer's medical qualifications;
searching means for searching the data storage means to select a volunteer from the search that is suited to provide the type of medical attention that is called for by the screening means, said volunteer being selected based upon their medical qualifications; and,
connecting means for establishing a connection over a telecommunications network between the individual and the selected volunteer such that they are able to interactively communicate with one another over the established connection.

11. The system of claim 10, wherein said screening means poses a series of medical questions to the individual and collects responses to the questions from the individual, wherein the type of medical attention that is called for is determined based upon the collected responses.

12. The system of claim 11, wherein the collected responses are communicated by the system to the selected volunteer to which the individual is connected.

13. The system of claim 11, wherein the screening means includes an expert system that automatically develops a diagnosis based upon the collected responses, said diagnosis being communicated by the system to at least one of the individual and the selected volunteer to which the individual is connected.

14. The system of claim 10, wherein the data storage means further contains contact information for each volunteer.

15. The system of claim 10, wherein each of the plurality of volunteers selectively enables and disables their own eligibility to be selected by the searching means.

16. The system of claim 15, wherein to enable their eligibility to be selected by the searching means a volunteer logs-in to the system and to disable their eligibility to be selected by the searching means a volunteer logs-out of the system.

17. The system of claim 10, wherein the data storage means also records a status indicator for each volunteer that shows them as one of logged-in or logged-out.

18. The system of claim 10, wherein the access means includes an interactive voice response subsystem that collects input from and provides output to the individual accessing the system.

19. The system of claim 10, wherein the telecommunications network is an Internet Protocol (IP) network.
